(19) United States
(12) Patent Application Publication
(10) Pub. No.: US 2006/0111641 A1
(43) Pub. Date: May 25, 2006

(54) System and method for ICG recording and analysis

(75) Inventors: Loren A. Manera, Laguna Hills, CA (US); Kenneth L. Burns, Laguna Hills, CA (US)

Correspondence Address: Crockett & Crockett, Suite 400, 24012 Calle De La Plata, Laguna Hills, CA, 92653, US

(73) Assignee: Applied Cardiac Systems, Inc.

(21) Appl. No.: 10/993,582

(22) Filed: Nov. 19, 2004

(51) Int. Cl. A61B 5/04

(52) U.S. Cl. 600/513

(57) ABSTRACT

An ICG control system for improved performance of Impedance Cardiography and improved reporting of the hemodynamic parameters obtained during an Impedance Cardiography (ICG) session. When the ICG control system is used, impedance waveforms, ECG Waveforms, and hemodynamic parameters are recorded continuously for each session. The parameters and/or pictorial representations are stored in a computer memory for recall, and the parameters may be recalled in formats particularly conducive to assessing and managing a patient's hemodynamic status.

### FIELD OF THE INVENTIONS

The inventions described below relate the field of Impedance Cardiography.

### BACKGROUND OF THE INVENTIONS

Impedance cardiography (ICG) is a technology that calculates changes in blood volume and flow velocity over time based on thoracic impedance measurements. ICG utilizes external electrodes to input a high frequency, low amplitude current and measure electrical impedance changes in the thorax. These external electrodes are typically placed on the neck and thorax of the patient. The impedance measurements are used to track volumetric changes such as those occurring during the cardiac cycle. Impedance (Z) changes are generated by blood volume and flow velocity increases and decreases in the ascending aorta during systole and diastole. Impedance to electrical current decreases during systole due to increased blood volume and flow velocity. The pulsatile impedance changes directly reflect blood flow leaving the heart and left ventricular function. The base thoracic impedance (Zₒ), pulsatile impedance/time changes (dZ/dt), and ECG are used to calculate stroke volume, cardiac output, and contractility parameters.

Many hemodynamic parameters can be ascertained with the use of ICG. Some of these parameters include: Cardiac Output (CO) which is the volume of blood flow per minute from the left ventricle; Stroke Volume (SV) which is the volume of blood ejected per beat from the left ventricle; System Vascular Resistance (SVR) which is the afterload resistance to left ventricle ejection; Change in Impedance/Time (dZ/dt) which is the magnitude and rate of change of impedance and is a direct reflection of the force of left ventricular contraction; Pre-ejection Period (PEP) which is the period between the start of a heartbeat and the ventricular contraction (measured from the onset of ventricular depolarization to the beginning of mechanical contraction); Ventricular Ejection Time (VET) which is the systolic time interval between the aortic valve opening to aortic valve closing; Acceleration Contractility Index (ACI) which is a direct reflection of myocardial contractility calculated from the rate of change of blood flow and peak acceleration in the ascending aorta; Left Cardiac Work Index (LCWI) which is a reflection of myocardial oxygen demand; and Thoracic Fluid Content (TFC) which is representative of total fluid volume in the chest, comprised of both intra-vascular and extra-vascular fluid. All of these measurements can be gathered continuously and non-invasively with ICG. During an ICG evaluation session, a patient's ICG waveform and hemodymanic parameters are examined.

Though ICG technology has improved since it was first developed by NASA in the 1960's, the user interface and control systems of ICG equipment have not improved at the same pace. Currently, ICG systems show real-time diagnostic information of the patient. A technician typically prints the diagnostic information such as waveform and hemodynamic parameters from the ICG system as it occurs. The printout usually provides only a few seconds of data, selected subjectively and without reference to historical data, during a session. In current ICG systems, entire ICG sessions are not constantly monitored and recorded in real time. The technician must decide when, during an ICG session, to begin printing the ICG report of the patient's parameters. Once printed, the technician then provides this printout to the doctor for evaluation and management of the patient's hemodynamic status. This report printing process can lead to missing diagnostic information that is vital to assessing and managing a patient's hemodynamic status. The technician may miss printing out important data or the patient's anomaly may not occur during the time when the technician is printing the report.

In addition to the deficiencies in recording and recalling of patient hemodynamic parameters, current ICG systems lack a user interface that allows the technician or doctor to record patient demographic information. ICG evaluation data is currently printed out and placed in a patient's medical record or file. There is no method to record patient information directly in an ICG report or view it in an ICG System. Information such as a patient's cardiologist, medications, drug allergies, indicators, or symptoms can be crucial in assessing and managing a patient's hemodynamic status.

Finally, current ICG systems fail to provide technical assistance and support to the technician or doctor. Presently, support information such as definitions for abbreviations used in ICG sessions and diagnostic information for disease symptoms are not provided in ICG systems. Doctors and technicians assess and manage a patient's hemodynamic status by reviewing the data provided in ICG reports. Presently, medical personnel must look over several ICG sessions and to other references in order to obtain information related to interpreting ICG data and data related to patient diagnosis. Quick references for use by the technician or doctor are simply not available in ICG systems.

### SUMMARY

The systems and method described below provide for improved performance of Impedance Cardiography and improved reporting of the hemodynamic parameters obtained during an Impedance Cardiography (ICG) session. When the ICG control system is used, impedance waveforms, ECG Waveforms, and hemodynamic parameters are recorded continuously for each session. The parameters and/or pictorial representations are stored in a computer memory for recall, and the parameters may be recalled in formats particularly conducive to assessing and managing a patient's hemodynamic status.

The ICG control system is used in conjunction with an Impedance Cardiography Device. The system includes several sets of electrodes for use on the neck and thorax of the patient, a current source, an ECG monitor, and a computer to control the operation of the system and record system parameters and hemodynamic parameters. The system provides ECG waveforms, ICG waveforms, data, hemodynamic parameters, the ability to review ICG sessions, patient demographic information, and technical support to users.

### BRIEF DESCRIPTION OF THE DRAWINGS

FIG. 1 illustrates an Impedance Cardiogram System (ICG).

FIG. 2 illustrates conventional strips used by doctors to review ICG evaluation sessions.

FIG. 3 illustrates a portion of the new report for the ICG control system with a user interface.

FIG. 4 illustrates a narrative report of the ICG control system having a user interface, a hemodynamic narrative, and an ectopic narrative.

FIG. 5 illustrates a full disclosure portion of the new report provided by the ICG control system.

FIG. 6 illustrates selected time frame strips of the ICG control system session.

FIG. 7 illustrates the trend analysis feature of the ICG control system identifying an ectopic.

FIG. 7A shows the trend analysis feature of the ICG control system identifying an ectopic in more detail.

FIG. 8 illustrates a hemodynamic trend report generated by the trend analysis feature of the ICG control system analyzing trends over a 24 hour time period.

FIG. 9 illustrates a minute-by-minute hemodynamic trend report generated by the trend analysis feature of the ICG control system.

FIG. 10 illustrates the technical support feature of the ICG control system.

### DETAILED DESCRIPTION OF THE INVENTIONS

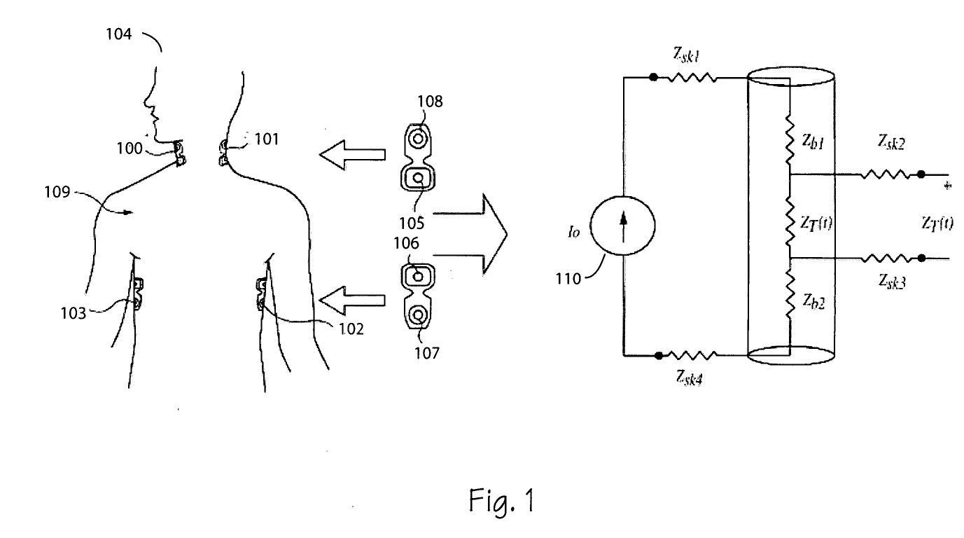

FIG. 1 illustrates an Impedance Cardiogram System (ICG) installed. In an ICG system, electrode arrays 100, 101, 102, 103 are placed on a patient 104. An electrode array comprises a stimulation electrode terminal 105, 106 and a measurement electrode terminal 107, 108. Electrode arrays are typically applied above and below the thorax 109 of a patient 104. An AC current from a current source 110 is supplied to the stimulation electrode terminals 105, 106. As shown in FIG. 1, current flows from each stimulation electrode terminal 105, 106 through each constant skin impedance, Zₛₖ₁ or Zₛₖ₄, each constant body tissue impedance, Z_{b1} or Z_{b1}, and each constant skin impedance, Zₛₖ₂ or Zₛₖ₃, to each measurement electrode terminal 107,108. The voltages at the measurement electrode terminals 107,108 are measured and input to a differential amplifier to obtain the differential voltage, V_{T} (t). The thoracic impedance of the patient, Z_{T} (t), is then obtained from these measurements.

As shown in FIG. 1, two sets of electrode arrays may be used to monitor the impedance associated with the left and right portion of the thorax 109. When eight electrode terminals (four arrays 100, 101, 102, 103) are used in this manner, the four measurement arrays are also used to obtain an electrocardiogram (ECG). The resulting electrocardiograms are used to determine the heart rate and trigger measurements of VET within the ICG system.

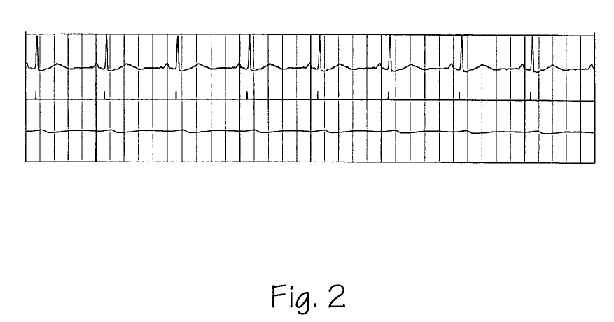

Conventionally, measurements from an ICG session have been monitored by reviewing selectively-generated strip charts, taken from a very small period within the ICG session. FIG. 2 illustrates a typical strip chart 113 provided to doctors to report the results of an ICG session. In the past, ICG strip charts have been generated by an operator, on the fly, during an ICG session. Each strip includes the patient's ECG and ICG wave form. Notes are hand-written by the operator. However, these strips represent short time-periods during the course of an ICG session. No record of the rest of the session is generated, and all the patient data from that session is lost.

The disclosed ICG control system captures ECG and ICG waveforms for an entire ICG session as well as multiple sessions and stores it in real-time in a computer. An ICG session is the time the patient is monitored by the ICG control system and the ECG and ICG data is recorded. Sessions may be of variable time frames ranging between seconds to days. Typically, an ICG session will last 5-7 minutes. The computer can have any memory device such as magnetic data storage, holographic data storage, optical data storage, or flash memory. The control system records the data for the entire ICG session, and, if desired, for multiple ICG sessions, for review and analysis by medical personnel. This data may also be transferred from the ICG control system to other computers, data bases, and servers.

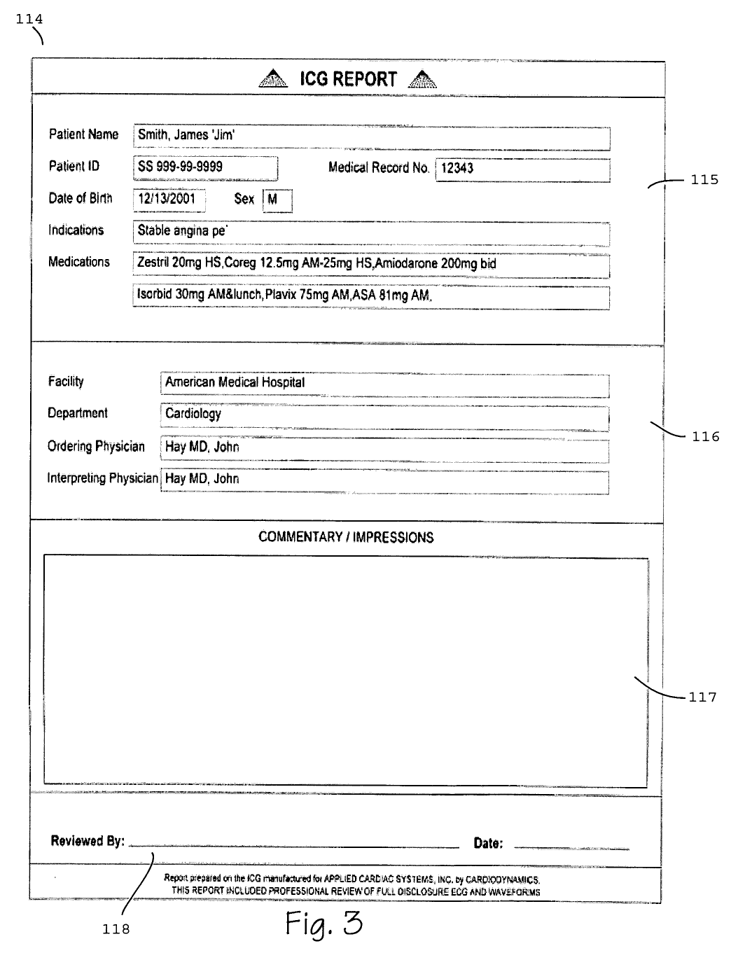

A portion of the new report with a user interface for the ICG control system is illustrated in FIG. 3. The ICG report 114 can be printed out or displayed on a computer. The report 114 contains a patient demographic field 115, a medical facility field 116, a commentary field 117, and a review field 118. A user of the ICG control system can record and display various types of information in the fields. The demographic field 115 enables a user to record and display patient information such as patient name, patient identification number, patient date of birth, sex, patient medications, and indications of an associated medical condition. The medical facility field 116 contains pertinent information relating to where the ICG session is taking place. This information may include the name of the medical facility, the department in that medical facility, the physician ordering the ICG, and the physician interpreting the ICG results. A commentary field 117 is also included in the ICG report. In this field, users can record miscellaneous information relating to the patient. A review field 118 is also found in the new report. If the report is printed out, a physician can simply sign the report and place it in a patient's file. However, since the ICG control system is digital, the system contains a digital signature for authorized personnel to certify they have reviewed the ICG report. This feature prevents unauthorized personnel from certifying the ICG or ECG data has been reviewed.

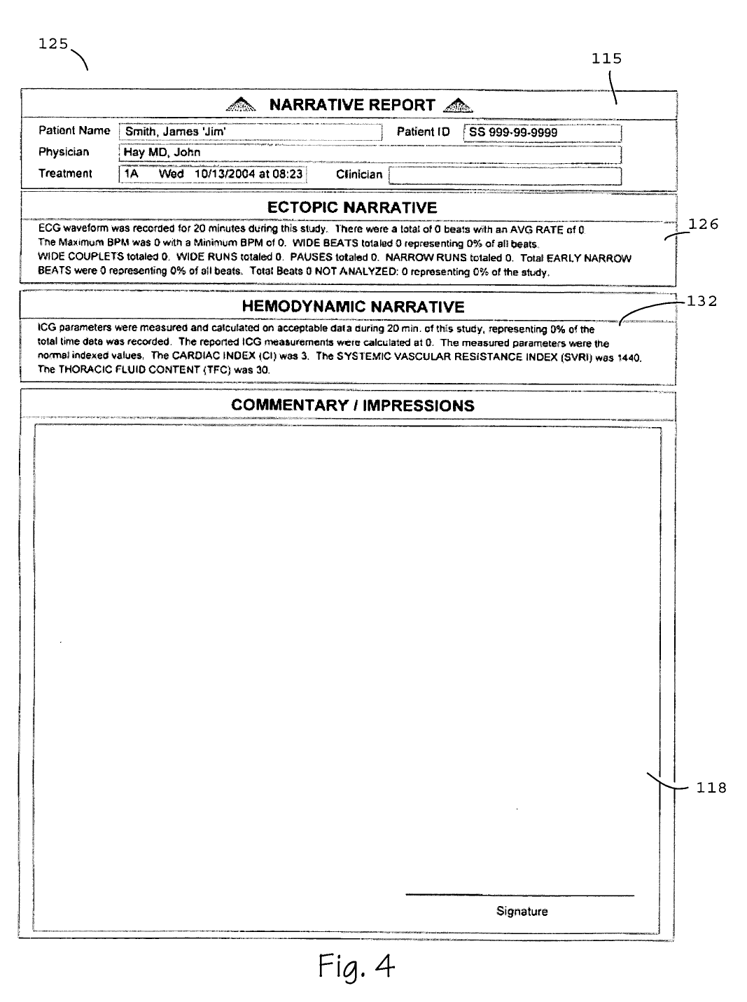

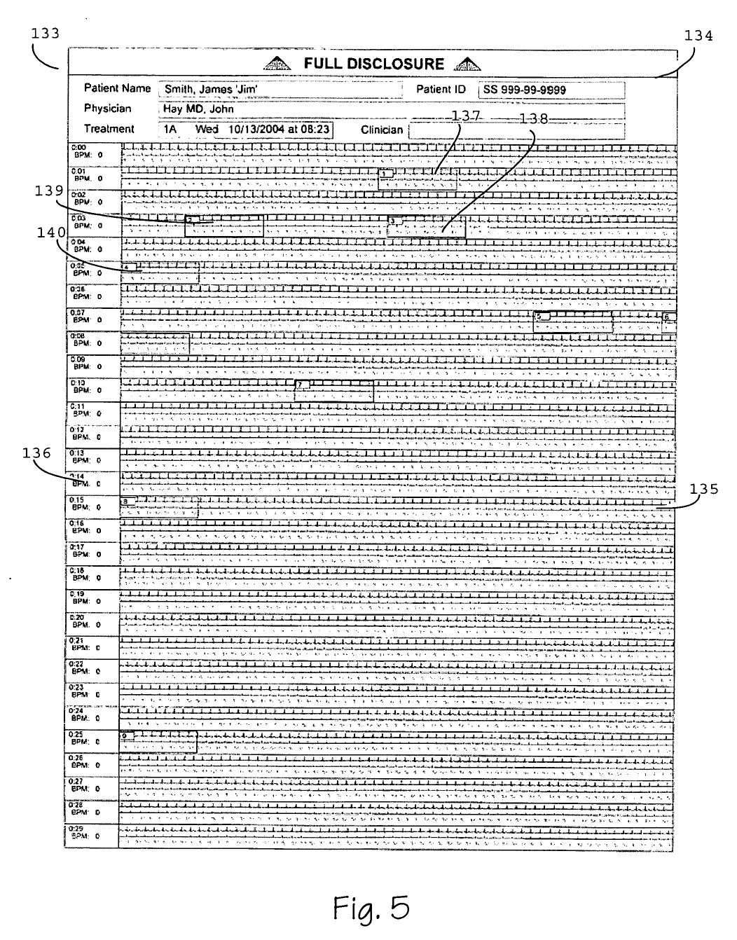

FIG. 4 illustrates a new narrative report of the ICG control system having, a graphical user interface, a hemodynamic narrative, and an ectopic narrative. Since the ICG control system continuously records data for an entire ICG session, more complete data for assessing and managing a patient's hemodynamic status can be obtained. The narrative report 125 may be provided in printed form or on a computer display. The narrative report is derived from data recorded during the ICG session and specifically selected by the user for evaluation. A full disclosure of an entire ICG session is shown in FIG. 5. The ICG control system user may select the entire ICG session or discrete time slips from the entire ICG session for reporting.

The narrative report 125 is presented in tabular format. The patient demographic field 125 can record and display various types of patient information. Information such as patient name, patient identification number, the patient's physician, the treatment time, and the ICG technician performing the ICG can all be recorded and presented in this tabular field. Information recorded in the ICG report in FIG. 3 can automatically be transferred to the respective fields in the narrative report by the control system.

An ECG ectopic narrative field 126 displays ECG data being evaluated by the narrative report 125 in an easy-to-read format for the user. This tabular field displays ECG information recorded during an ICG session such as total number of Heart Beats, Average Rate, Maximum Beats per Minute (BPM), number of Wide Beats, the percentage of Wide Beats when compared to the Total Beats studied in the ICG session, the number of Wide Couplets, the number of Wide Runs, the number of Pauses, the number of Narrow Runs, the total number of Early Narrow Beats and its percentage of total beats. The ectopic narrative field also displays the total beats not analyzed by the report and what percentage the report covers of the entire ICG session.

The narrative report 125 contains a hemodynamic narrative field 127. The hemodynamic narrative field 127 displays ICG parameters being evaluated by the narrative report 125 in an easy-to-read format for the user. Information found in the hemodynamic narrative field may include the total recording time of the ICG session, the percentage of the total ICG session time being evaluated by the report, and hemodynamic parameters such as Stroke Volume (SV), System Vascular Resistance (SVR), Change in Impedance/Time (dZ/dt), Pre-ejection Period (PEP), Ventricular Ejection Time (VET), Acceleration Contractility Index (ACI), Left Cardiac Work Index (LCWI), and Thoracic Fluid Content (TFC).

FIG. 5 illustrates a full disclosure portion 128 of the new report provided by the ICG control system. This report may be provided in printed form or on a computer display. The ICG control system collects and stores all measured data collected from the patient and the ICG system during every ICG session.

FIG. 5 shows a thirty-minute portion of the data (the entire report to the session includes two pages) in a formatted table 129. As shown in the figure, full ECG and ICG data is recorded continuously for the entire ICG session. The ECG and ICG data recorded by the control system includes ECG Waveforms and ICG Waveforms. From the data and waveforms, hemodynamic parameters such as Cardiac Output (CO), Stroke Volume (SV), System Vascular Resistance (SVR), Change in Impedance/Time (dZ/dt), Pre-ejection Period (PEP), Ventricular Ejection Time (VET), Acceleration Contractility Index (ACI), Left Cardiac Work Index (LCWI), and Thoracic Fluid Content (TFC) are derived and recorded. Also, from the waveforms and data, ECG parameters such as total number of Heart Beats, Average Rate, Maximum Beats per Minute (BPM), Wide Beats, Total Beats made in the ICG session, Wide Couplets, Wide Runs, the number of Pauses, Narrow Runs, and Early Narrow Beats are derived and recorded. Patient demographic information can be displayed and recorded in a patient demographic display field 115 of the formatted table.

The ECG data and ICG data are presented in trace format in numerous trace display fields 130. The session time (in minutes) and heart rate (in BMP) are reported in tabular form in tabular data display fields 131. One trace display field and one tabular data display field is provided for each minute of an ICG session. Each trace field contains an ECG trace and an ICG trace. These graphical elements are presented in synchronized relationship

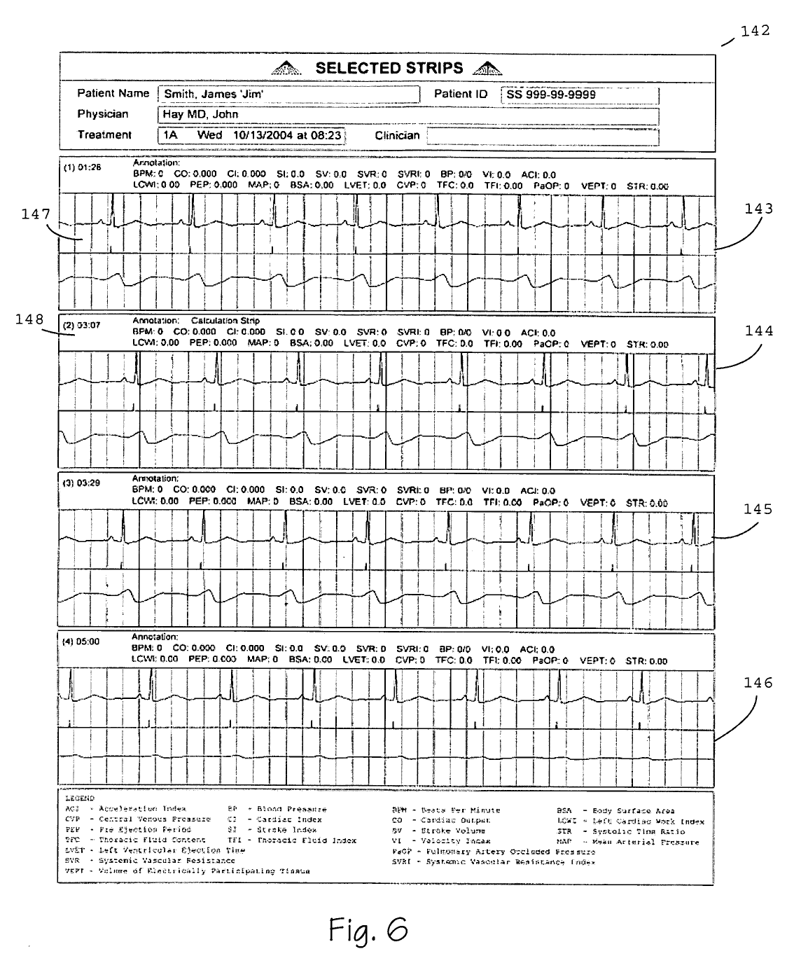

An operator reviewing the report of FIG. 5 may review the entire report, including data for the entire ICG session, and appreciate data trends that are necessary in assessing and managing a patient's hemodynamic status. The operator may manipulate shaded cursors 132, 133, 134, and 135 to select time periods of particular interest for expansion into a larger format shown in FIG. 6. The cursors are labeled as cursor 1, 2, 3, and 4 (or other distinct indicia) so that they may be correlated to the selected strips generated for FIG. 6. The cursors are depicted as shaded blocks or windows, but they may be provided in any other form that allows operator manipulation and location of the cursor to be used as an input of the desired time period to be expanded

FIG. 6 illustrates a selected strips report generated by the system based on discrete time periods selected by the user through manipulation of strip selecting cursors 132, 133, 134, and 135. The selected strips report 137 presents the time periods selected in FIG. 5. Typically, the selected strip report 137 can show five (5) to eight (8) minutes of the total data stored by the ICG control system. However, the strip report 137 could be designed to select and show larger or smaller discrete time frames of the ICG session. The selected strips 138 139 140 141 are presented in larger scale, for closer analysis of the relationship between the ECG, ICG, hemodynamic parameters, and ECG parameters. The selected strips include a trace display field 142 and a tabular data field 143. As can clearly seen in the Figure, each selected strip 138 139 140 141 presents the time period during the ICG session corresponding to the strip, a strip number, and the patient's ECG and ICG data for the selected strip's time period. This data may include Acceleration Index (ACI), Cardiac Output (CO), Cardiac Index (CI), Stroke Index (SI), Stroke Volume (SV), System Vascular Resistance (SVR), System Vascular Resistance Index (SVRI), Blood Pressure (BP), Velocity Index (VI), Acceleration Index (ACI), Pre Ejection Period (PEP), Mean Arterial Pressure (MAP), Body Surface Area (BSA), Left Ventricular Ejection Time (LVET), Central Venous Pressure (CVP), Thoracic Fluid Content (TFC), Thoracic Fluid Index (TFI), Pulmonary Artery Occluded Pressure (PaOP), Volume of Electrically Participating Tissue (VEPT), and Systolic Time Ratio (STR). An operator can also review the ECG and ICG waveform captured during the selected strip's time period.

Since a complete ICG session or multiple ICG sessions is recorded for the patient or multiple patients in a database, the ICG control system can evaluate trends found in the ECG and ICG parameters of the patient occurring over time using a trend analysis feature. The trend analysis feature can assist medical personnel by analyzing ECG and ICG parameters of the patient and provide possible diagnosis corresponding to the hemodynamic parameter trends occurring in the patient. The trend analysis tool may also be used determine treatment options respective to the diagnosis. The ICG system creates a report enabling the physician to review patient progress on a minute, hour, daily, or monthly basis. The control system allows the user to select individual ICG sessions, multiple ICG session, or several discrete time frames of an ICG session or multiple session for analysis. The trends provided by the report can show progress of patients being treated with medical or other therapy. The ICG control system is able to report data outside of the normal range. The system generates the trends that will assist the physician with treatment strategy, assessment of heart failure treatment, ruling out cause of dyspnea, determination of therapy direction, determination of causes of hypertension, and optimization and validation of medication prescription. Storage of ECG and ICG data over time for a patient or multiple patients allows for serial comparison of the data for patient diagnosis and medical research.

On screen review of all calculations show low, average and high standings unique to each patient over time. This can be used to monitor immediate effect of medical or other therapy as well as long-term response of cardiac therapy. The analysis feature can assist in the delivery of ECP therapy on high-risk patients as well. The ICG control system annotates measurements that are low, within normal limits and high.

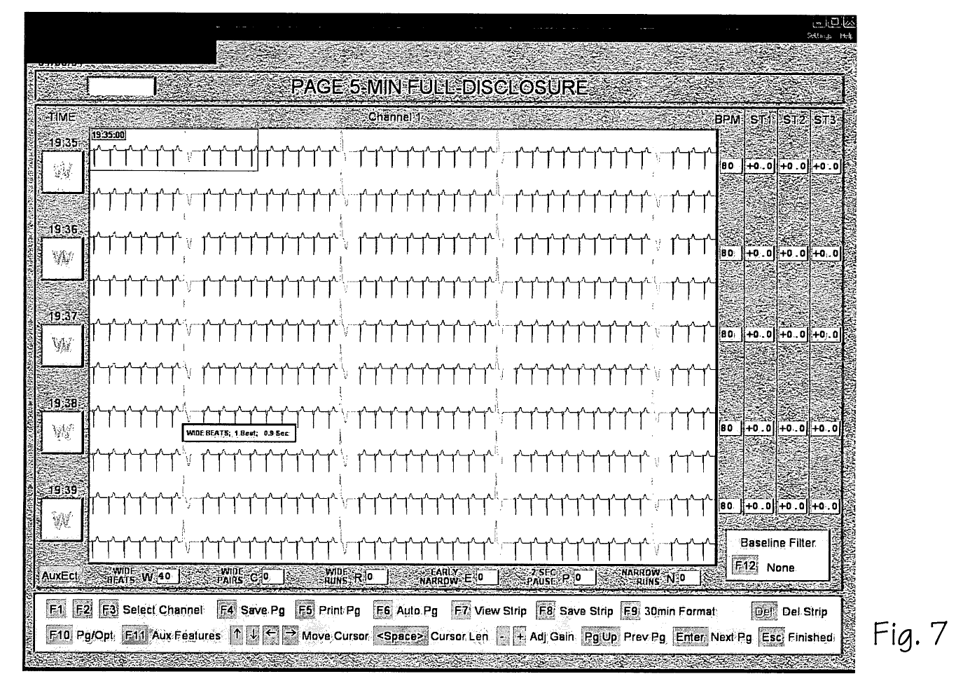

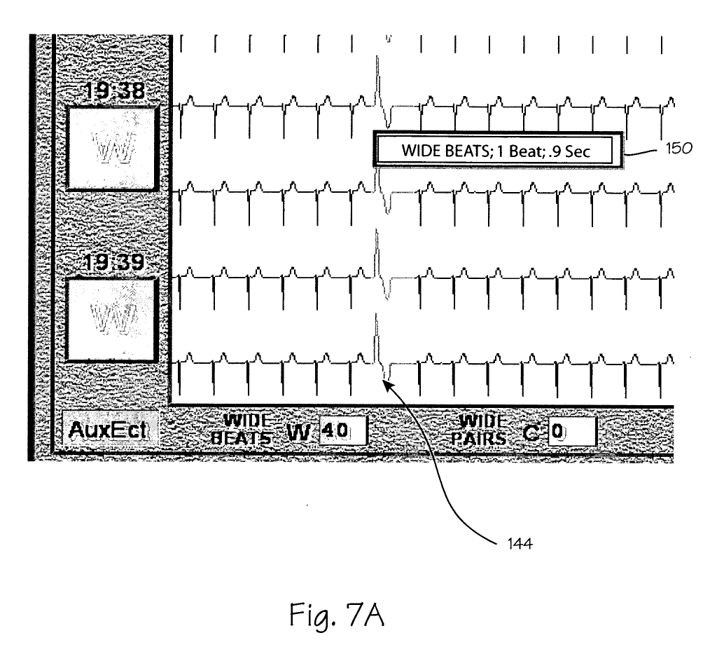

FIG. 7 illustrates the trend analysis feature of the ICG control system in use during a five minute full disclosure report. Because ECG and ICG data from a patient is continuously recorded by the ICG control system, the system has the capability to perform an Arrythmia analysis on the ECG and identify corresponding ectopics. This allows the technician to capture the ICG hemodynamic parameters when the ECG is normal allowing accurate ICG calculations. FIG. 7A illustrates a more detailed view of the trend analysis feature shown in FIG. 7. In this figure, the ICG control system analysis feature identifies wide beats 144 and the technical support display field 150 displays the identification of the ectopic (a wide beat), the number of wide beats (1), and the time of the beat (0.9 seconds).

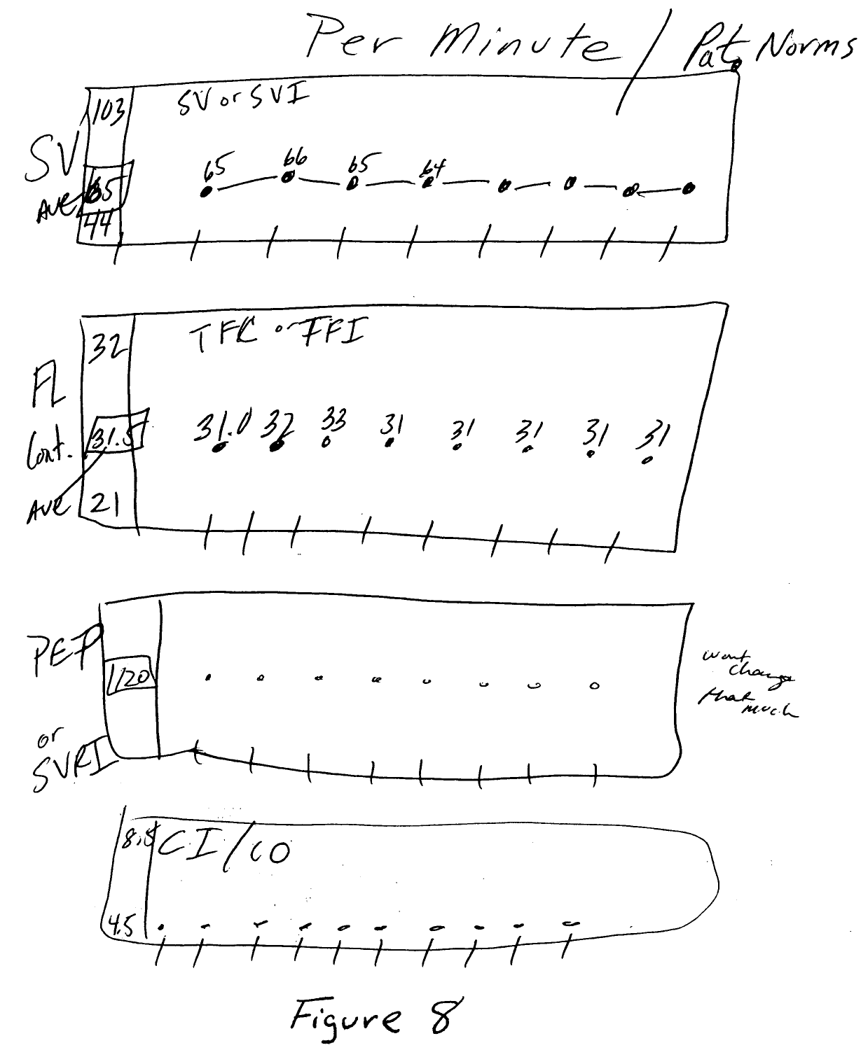

FIG. 8 illustrates a report generated by the trend analysis feature of the ICG control system. This report displays a patient's hemodynamic trends over several ICG sessions for a cumulative time of twenty-four hours. In FIG. 8, trends for a patient's Stroke Volume (SV), Thoracic Fluid Content (TFC), System Vascular Resistance Index (SVRI), and Cardiac Output (CO) are displayed.

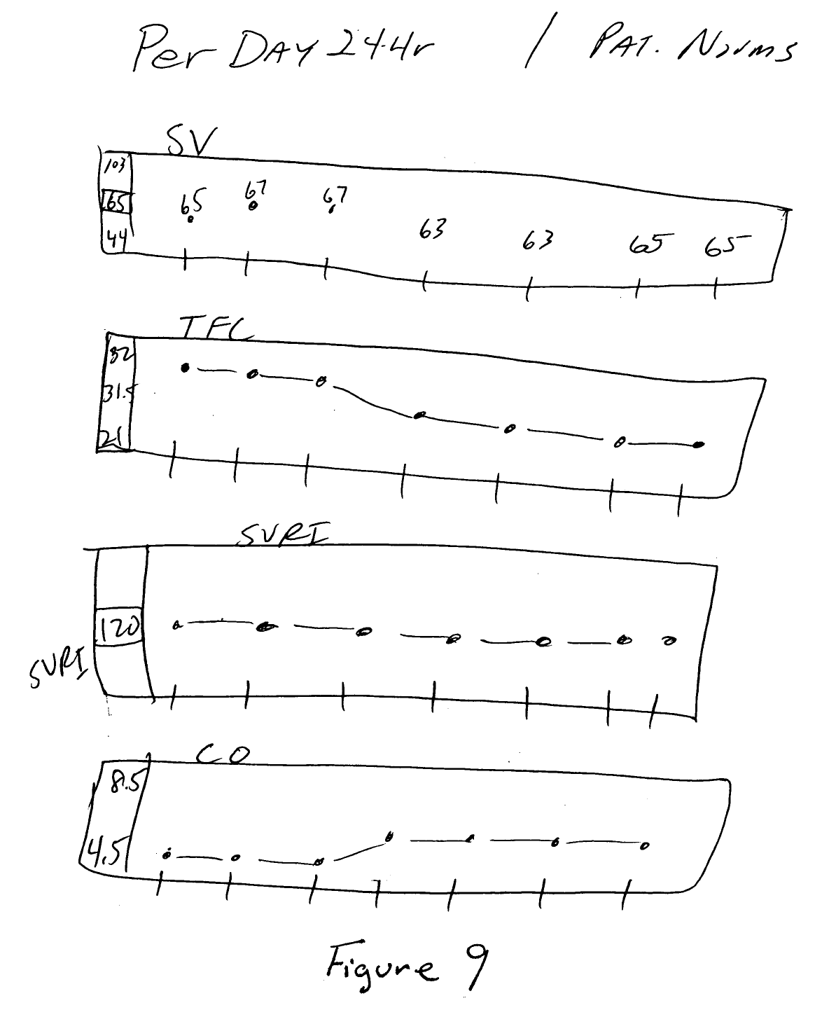

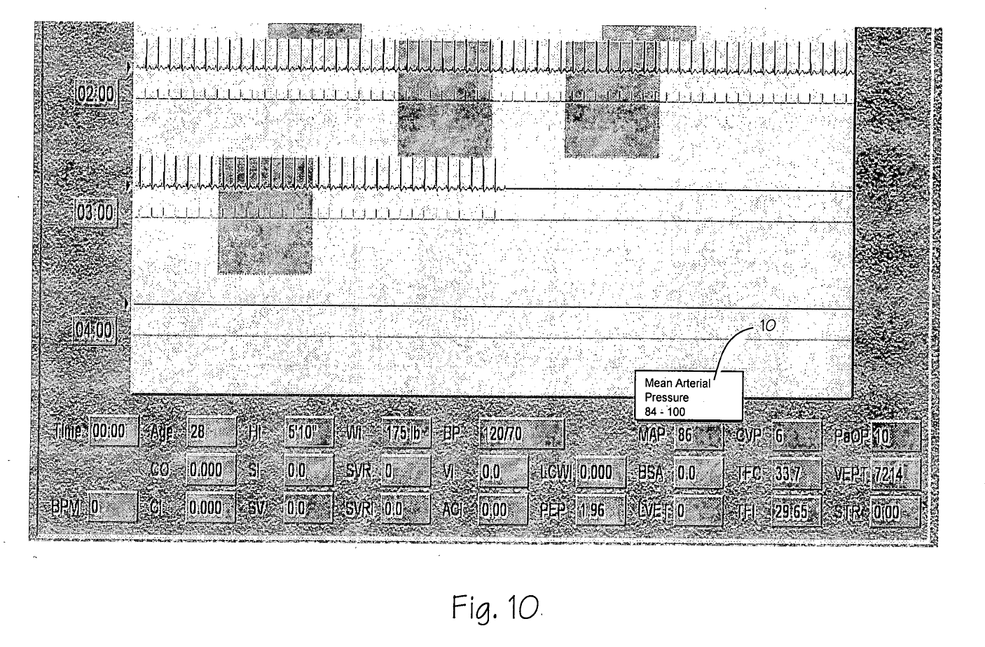

FIG. 9 further illustrates a report generated by the trend analysis feature of the ICG control system. This report displays a patient's hemodynamic trends over a minute-by-minute period. In FIG. 9, trends for a patient's Stroke Volume (SV), Thoracic Fluid Content (TFC), System Vascular Resistance Index (SVRI), and Cardiac Output (CO) are displayed. The patient's measurements for each parameter are shown. In addition to being able to review selected strips 138 139 140 141 from an ICG session or multiple sessions, the ICG control system provides a detailed technical support feature to its users. The technical support feature can display ECG or ICG related information to the ICG control system's users. This information may include more detailed definitions of hemodynamic parameters, acceptable ranges of hemodynamic parameters, current status of hemodynamic parameters, diagnosis options, and treatment options. FIG. 10 shows the technical support feature in use in the ICG control system. The user selects an ICG term of interest, in this case MAP, using a pointer 149, cursor, or other indicia. A technical support field 150 displays a definition of the term selected as well as acceptable hemodynamic levels for the respective parameter. Here, MAP is defined as Mean Arterial Pressure and an acceptable range for a patient is 84-100.

Thus, while the preferred embodiments of the devices and methods have been described in reference to the environment in which they were developed, they are merely illustrative of the principles of the inventions. Other embodiments and configurations may be devised without departing from the spirit of the inventions and the scope of the appended claims.

We claim:

1. An ICG control system comprising:
a current source;
one or more electrode arrays with said electrode array comprising a stimulation electrode terminal and a measurement electrode terminal;
an ICG monitoring system operable to continuously detect the patient's ICG and transmit the ICG data;
an ECG monitoring system operable to continuously detect the patient's ECG and transmit ECG data; and
a computerized control system operable to control the current source and the electrode arrays, receive and record ECG data from the ECG monitoring system, and receive and record ICG data from the ICG monitoring system;
wherein the computerized control system is further programmed to:
present a first display comprising fields for entry of patient data;
record hemodynamic parameters for substantially the entirety of an ICG session;
present a second display comprising one or more trace data fields and present an ECG trace generated from the ECG data and present an ICG trace generated from the ICG data; and
analyze hemodynamic parameters and ECG data recorded during the ICG session for occurring trends.

2. The system of claim 1 wherein:
the control system is further programmed to display substantially continuous ECG data and substantially continuous ICG data for the entirety of the ICG session in the second display, in multiple display pages, with each display page displaying portions of a single record for the entire ICG session.

3. The system of claim 1 or 2 wherein the control system is further programmed to display a cursor in relationship to the trace data field and interpret operator manipulation of the cursor as input as to desired small time periods represented within the trace data field, and thereafter present a third display comprising an enlarged trace data field covering a time period consisting of the desired small time period.

4. The system of claim 1, 2, or 3 wherein the control system is further programmed to present a plurality of cursors in logical proximity to the ICG trace, and interpret operator manipulation of the cursors as input as to operator determined.

5. The system of claim 4 wherein the control system is further programmed to present hemodynamic parameters of the patient respective to the small time periods selected by the operator in the enlarged trace data field of the third display.

6. The system of claim 4 wherein the control system is further programmed to present ECG data of the patient respective to the small time periods selected by the operator in the enlarged trace data field of the third display.

7. The system of claim 1 wherein the control system is further programmed to present a technical support field.

8. The system of claim 7 wherein the technical support field contains definitions of hemodynamic parameters.

9. The system of claim 7 wherein the technical support field contains clinical diagnosis options reflective of the hemodynamic parameters or ECG data of the patient.

10. The system of claim 7 wherein the technical support field contains treatment options respective of the hemodynamic parameters or ECG data of the patient.

11. A method for controlling and evaluating an ICG session comprising:
recording substantially continuous ICG data for an entirety of ICG session;
recording substantially continuous ECG data for the entirety of the ICG session;
displaying trace fields of ICG data and ECG data representing data recorded for the entirety of an ICG session;
selecting small time periods of the ICG session; and
displaying an enlarged trace data field of the small time period with the enlarged trace field containing hemodynamic and ECG data respective to the small time period.

12. The method of claim 11 further comprising analyzing the ECG data and ICG data for an Arrhythmia.

13. The method of claim 11 further comprising analyzing the ECG data and ICG data for medical diagnosis.

14. The method of claim 11 further comprising displaying a technical support field.

15. The method of claim 11 further comprising analyzing the ECG data and ICG data for data trends.
